(19) United States
(12) Patent Application Publication
(10) Pub. No.: US 2018/0052106 A1
(43) Pub. Date: Feb. 22, 2018

(54) DUAL DETECTION SCHEME FOR DNA SEQUENCING

(71) Applicants: Kerry Gunning, San Diego, CA (US); Donald Ackley, Cardiff, CA (US); Chan-Long Shieh, Paradise Valley, AZ (US)

(72) Inventors: Kerry Gunning, San Diego, CA (US); Donald Ackley, Cardiff, CA (US); Chan-Long Shieh, Paradise Valley, AZ (US)

(21) Appl. No.: 15/239,032

(22) Filed: Aug. 17, 2016

(51) Int. Cl. G01N 21/64; C12Q 1/68; G01N 27/414

(52) U.S. Cl. CPC G01N 21/6428; C12Q 1/6869; G01N 2201/062; G01N 2021/6439; G01N 27/4145

(57) ABSTRACT

Apparatus for fluorescent and ion sensing of DNA nucleotide incorporation events including DNA nucleotide incorporation structure designed to have sequencing primers bonded to a surface for the incorporation of DNA nucleotides thereon. At least some of the DNA nucleotides having a fluorescent label. A photodiode positioned adjacent the incorporation structure and an illumination device positioned adjacent the DNA nucleotide incorporation structure to illuminate DNA nucleotides incorporated onto the sequencing primers. The illumination device exciting the fluorescent labels when incorporation occurs and the photodiode positioned to sense the excited fluorescent labels. Ion sensing apparatus positioned adjacent the DNA nucleotide incorporation structure including a metal oxide thin film transistor with a gate electrically coupled to receive an electrical signal indicative of ion emissions produced by the DNA nucleotide incorporated onto DNA target fragments or sequencing primers.

### FIELD OF THE INVENTION

This invention generally relates to DNA sequencing and more specifically to fluorescent and chemical sensing of nucleotide incorporation events.

### BACKGROUND OF THE INVENTION

To carry out the sequencing of the human genome, the DNA (deoxyribonucleic acid) is cut into short fragments, the fragments are sequenced simultaneously and the data may then be assembled using sophisticated computer technology. DNA sequencing is the process of determining the precise order of nucleotides (thymine, adenine, guanine, and cytosine) within a DNA molecule. DNA sequencing by synthesis is commonly achieved using one of two sensor modalities to monitor nucleotide incorporation. The two sensed modes or modalities are optical detection of fluorescently tagged nucleotides and the use of ion selective field effect transistors (ISFETs) to detect hydrogen ions that are released when a nucleotide is incorporated onto a target DNA fragment.

Typically, the optical detection schemes incorporate complicated optical instrumentation to scan across large substrates. The four nucleotides are distinguished by assigning a different wavelength fluorophore to each nucleotide. By assigning different target DNA fragments to each site on a substrate and monitoring fluorescence color, the identity of each nucleotide that incorporates onto the target fragment may be determined. This system has the advantage that all nucleotides may be introduced simultaneously, but requires a complex optical system to monitor four colors simultaneously across a large substrate. This system also requires the use of modified polymerases that have been selectively engineered to accommodate the industry standard dual-modified nucleotides. The dual-modified nucleotides are independently tagged with a fluorescent moiety and a 3′-block to prevent subsequent nucleotide polymerization.

Alternatively, the incorporation of nucleotides onto the target fragment may be determined by monitoring a local pH change that occurs as hydrogen ions are released during a nucleotide base incorporation event. Typically, target DNA fragments are distributed onto beads and biologically amplified on the beads using PCR (Polymerase Chain Reaction). The beads are then loaded onto an array of ISFETs such that one bead is incorporated into one well on top of each ISFET. The four nucleotides, one at a time, are then flowed across the array in serial fashion, and the pH change upon nucleotide incorporation is monitored at each pixel (each ISFET of the array) to determine to which target or pixel a nucleotide base has incorporated. This system has the advantage that it does not require a complex optical system to monitor the incorporation, but conversely the nucleotides must be flowed serially across the ISFET array and there are issues in discerning homopolymer regions (i.e., a region in the target DNA fragments or strands with a number of the same bases occurring in a row). Another advantage is that standard polymerases can be used in the sequencing reaction since standard nucleotides are used for this scheme.

In order to simplify the optical system associated with the fluorescent detection process, schemes have been proposed which only use a single color fluorescence for detection of all four nucleotides. The most recently announced one-channel chemistry scheme (from Illumina) describes the following steps: “thymine will have a permanent fluorescent label. Adenine will have the same fluorescent label, but that dye will be removable. Guanine will be permanently dark. And, cytosine will start dark but will be tagged so that a dye can be added to it.” Illumina has then described how this scheme would work to read the DNA. “Essentially, in a first image of the four nucleotides, A and T are both labeled and detectable. Then, in the second image, the dye is cleaved from A and added to C. In the second image, only C and T fluoresce. By combining the information from the two images, all four bases are easily discriminated.”

The issue with this scheme for clinical applications is that the incorporation of the nucleotide guanine is a null event. That is, the site will be dark if there is a guanine incorporation event, or if there is no incorporation at all. This potentially introduces errors into the detected DNA sequences and is unlikely to receive FDA approval.

It would be highly advantageous, therefore, to remedy the foregoing and other deficiencies inherent in the prior art.

Accordingly, it is an object of the present invention to provide a new and improved detection process for DNA sequencing.

It is another object of the present invention to provide a new and improved detection process for DNA sequencing incorporating both an optical detection process and a process of detecting hydrogen ions that are released when a nucleotide is incorporated onto a target DNA fragment.

### SUMMARY OF THE INVENTION

The desired objects of the instant invention are achieved in accordance with apparatus for fluorescent and ion sensing of DNA nucleotide incorporation events including DNA nucleotide incorporation structure designed to have sequencing primers bonded to a surface for the incorporation of DNA nucleotides thereon, with at least some of the DNA nucleotides having a fluorescent label. A photodiode is positioned adjacent to the incorporation structure and an illumination device positioned in proximity to the DNA nucleotide incorporation structure to illuminate DNA nucleotides incorporated onto the sequencing primers. The illumination device excites the fluorescent labels when incorporation occurs and the photodiode is positioned to sense the fluorescence from the excited labels. Ion sensing apparatus is additionally positioned adjacent to the DNA nucleotide incorporation structure including a metal oxide thin film transistor with a gate electrically coupled to receive an electrical signal indicative of ion emissions produced by the DNA nucleotide incorporated onto DNA target fragments or sequencing primers.

The desired objects of the instant invention are also achieved in accordance with a method of fabricating apparatus for deoxyribonucleic acid (DNA) sequencing and more specifically for fluorescent and ion sensing of DNA nucleotide incorporation events. The method includes the steps of providing a substrate, fabricating either ion sensing apparatus including a metal oxide thin film transistor or an amorphous silicon photodiode on the substrate, and fabricating the other of the ion sensing apparatus and the amorphous silicon photodiode adjacent to the one fabricated on the substrate. The method also includes fabricating either a reservoir or a well overlying the amorphous silicon photodiode, and fabricating both the reservoir and the well with a transparent bottom and a sensing layer incorporated in the bottom. The sensing layer includes an ion sensing element positioned to sense ion emissions in the reservoir or the well and electrically coupling the sensing element to a gate of the metal oxide thin film transistor. Both the reservoir and the well are designed to have DNA target fragments or sequencing primers bonded to a surface for the incorporation of DNA nucleotides onto the DNA target fragments or sequencing primers, at least some of the DNA nucleotides having a fluorescent label. The method also includes a step of providing an illumination device positioned adjacent the reservoir or the well to illuminate DNA nucleotides incorporated onto the DNA target fragments or sequencing primers, the illumination device exciting the fluorescent labels when incorporation occurs with the photodiode positioned to sense the excited fluorescent labels.

The desired objects of the instant invention are also achieved in accordance with a method of deoxyribonucleic acid (DNA) sequencing and more specifically fluorescent and ion sensing of DNA nucleotide incorporation events. The method includes the steps of: providing a sensing pad and bonding sequencing primers to a surface of the sensing pad; attaching target DNA fragments to the sequencing primers; attaching sequencing polymerase enzymes to the target DNA fragments; using the sequencing polymerase enzymes, incorporating matching nucleotides with the sequencing primers, whereas hydrogen ions are released upon incorporation of the matching nucleotides; attaching blocking molecules to the matching nucleotides and labeling the matching nucleotides with fluorophores; illuminating the attached and labeled target DNA fragments and sequencing primers to excite the fluorophores; sensing the release of hydrogen ions and the fluorescent emission of the fluorophores; cleaving the blocking molecules and the matching nucleotides from the sequencing primers; and repeating the steps of using, attaching blocking molecules, illuminating and sensing the release of hydrogen ions and the sensing of the fluorescence for additional sequencing events.

The desired objects of the instant invention are further achieved in accordance with a preferred embodiment of the above method wherein the steps of providing the sensing pad and sensing the release of hydrogen ions and the excitation of the fluorophores include providing apparatus for sensing both fluorescent and ion emissions during nucleotide incorporation events, the apparatus including an ion sensing metal oxide thin film transistor and an amorphous silicon photodiode on a common substrate, and one of a reservoir and a well overlying the amorphous silicon photodiode, both the reservoir and the well having a transparent bottom and a sensing layer incorporated in the bottom, the sensing layer including an ion sensing element positioned to sense ion emissions in the reservoir or the well, the sensing element electrically coupled to a gate of the metal oxide thin film transistor, and both the reservoir and the well having a surface that forms the sensing pad.

### BRIEF DESCRIPTION OF THE DRAWINGS

The foregoing and further and more specific objects and advantages of the instant invention will become readily apparent to those skilled in the art from the following detailed description of a preferred embodiment thereof taken in conjunction with the drawings, in which:

FIG. 1 is a simplified layer diagram illustrating a combined MOTFT ion sensitive and optical detection structure in accordance with the present invention;

FIG. 2 is a simplified layer diagram illustrating a combined MOTFT ion sensitive and optical detection structure with an a-Si photodiode on top of the ion sensitive MOTFT in accordance with the present invention;

FIG. 3 illustrates sequencing primers bound to a sensing pad surface, such as the sensing pad of structure 60;

FIG. 4 illustrates sequencing primers bound to a bead surface, such as the bead illustrated in the well of structure 10;

FIGS. 5 through 12 illustrate steps in a chemical process for improving detection of nucleotide incorporation;

FIG. 13 illustrates one photocleaving and illumination structure;

FIG. 14 illustrates another photocleaving and illumination structure; and

FIG. 15 illustrates the UV LED wavelength versus the green LED wavelength to show the separation.

### DETAILED DESCRIPTION OF THE DRAWINGS

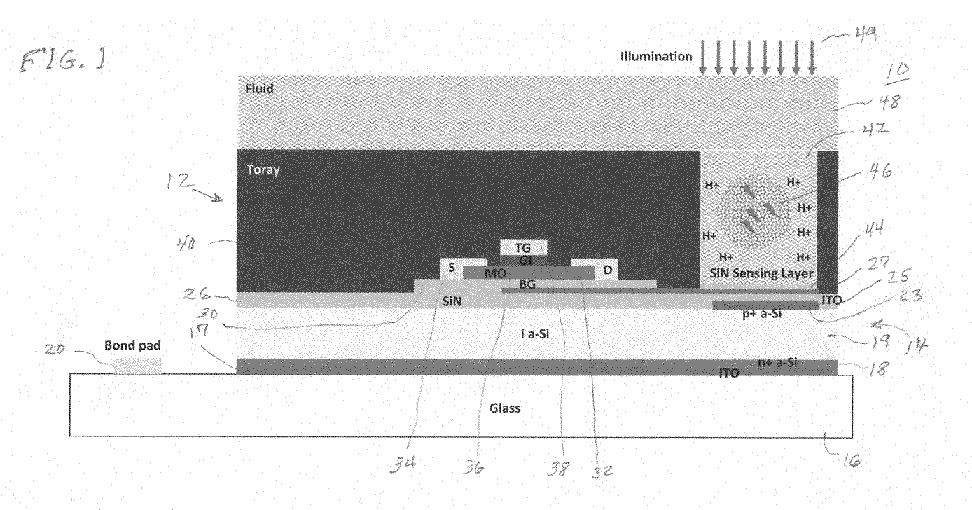

Turning to FIG. 1, a structure 10 is illustrated which includes and combines an ion sensitive MOTFT (metal oxide thin film transistor) 12 and an optical detection thin film photodiode 14. Structure 10 is fabricated on a substrate 16 which in this preferred embodiment is glass but could be other transparent material, such as plastic or the like. A lower contact 17 for photodiode 14 and structure 10 is deposited on the upper surface of substrate 16 and defines an area on which the combined MOTFT 12 and photodiode 14 are fabricated. Contact 17 may be any of the well-known conductive materials used in the semiconductor industry, such as indium-tin-oxide (ITO), Mo, Al, and the like. An n+ doped layer 18 of a-Si (amorphous silicon) is deposited on the upper surface of contact 17 and an intrinsic or insulating layer 19 of a-Si is deposited on the upper surface of layer 18, both of which extend substantially over the area or upper surface of contact 17. A bond pad 20 is positioned on the upper surface of substrate 16 to one side so as to be in an area not covered by contact 17 but which is readily accessible, along with an edge of contact 17, for easy interrogation of MOTFT 12 and photodiode 14, preferably, simultaneously or in a specific order on a single incorporation event.

A p+ doped layer 23 of a-Si is deposited on the upper surface of layer 19 adjacent an edge of layer 19 in a smaller area and forms amorphous silicon thin film diode 14 in combination with layers 19 and 18. A layer 25 of transparent conductive material, such as ITO, is deposited on the upper surface of layer 23 and provides an upper contact for photodiode 14. As will be explained in more detail below, photodiode 14 is positioned relative to MOTFT 12 so that a well can be formed adjacent to MOTFT 12 directly above and in light communication with photodiode 14.

A layer 26 of silicon nitride (SiN) is formed or deposited so as to extend over the entire area of layer 19, including the area covered by layer 23 and layer 25 overlying layer 23. A layer 27 of transparent conductive material (e.g. ITO) is deposited on the upper surface of SiN layer 26 so as to extend from an area above layer 25 to a mid-point in structure 10 where it serves as a bottom gate for MOTFT 12. A layer 30 of gate dielectric material, preferably a second layer of SiN, is deposited or formed over a portion of conductive layer 27 in the central area of structure 10. A layer 32 of semiconductor metal oxide is deposited/formed on the upper surface of gate dielectric layer 30 overlying a portion of bottom gate layer 27. Spaced apart source/drain contacts 34 are deposited/formed on semiconductor metal oxide layer 32. Optionally, an additional layer 36 of gate dielectric material is deposited/formed on the upper surface of semiconductor metal oxide layer 32 between source/drain contacts 34 and a metal top gate 38 is deposited/formed on the upper surface of gate dielectric layer 36 so as to define a channel area in semiconductor metal oxide layer 32. As will be understood, source/drain contacts 34 and top gate 38 include electrical connections (not shown) designed to electrically couple MOTFT 12 into external circuitry, such as a switch matrix or the like, and to bond pad 20. Also, lower gate layer 27 is coupled to conductive layer 25 so as to couple photodiode 14 into the circuit.

A thick layer 40 of insulating encapsulation material is deposited over MOTFT 12 and photodiode 14. As will be understood by artisans in the field, the insulating encapsulation material is selected to have a minimum and preferably no effect on both the electrical and chemical components of structure 10 and the subject tests. A well 42 is formed in layer 40 in overlying relationship with photodiode 14 and more specifically p+ doped a-Si layer 23. The horizontal extent of well 42 is slightly greater than the extent of p+ doped a-Si layer 23 and extends vertically into layer 40 to conductive layer 27 (which might operate for example as an etch-stop). A layer 44 of dielectric or insulating material is deposited in the bottom of well 42 to electrically insulate well 42 from conductive layer 27. All of the material between well 42 and photodiode 14 is generally referred to as the ‘bottom’ of well 42 for convenience. The overall size (depth and width) of well 42 is designed to receive therein a bead 46 with biologically amplified target DNA fragments distributed thereon. A fluid 48 is used to carry nucleotides serially into well 42 for testing purposes.

In operation, when a labeled nucleotide carried by liquid 48 into well 42 is incorporated into the target DNA fragments on bead 46, a fluorescence event will occur when bead 42 is illuminated by an illumination source 49. The presence or absence of fluorescence is sensed by photodiode 14 which appears as a signal on contact 17. Simultaneously, the incorporation of nucleotides onto the target fragment release hydrogen ions and produce a change in the pH of liquid 48 in well 42. The change in pH is sensed by a small change in voltage on conductive layer 27 connected to the bottom gate of MOTFT 12. The small change in voltage on the bottom gate acts similar to a bias so that a larger signal on the top gate is required to activate (i.e. turn ON or turn OFF) MOTFT 12. Thus, the small signal is essentially amplified which, depending upon the design and construction of MOTFT 12, can be as much as a factor of 10. As is well-known in the art, the degree of such charge amplification is determined by the relative capacitances of the top and bottom gates. Here it should be understood that through proper design and selection of materials, MOTFT 12 can be fabricated with extremely low leakage current and enhanced mobility of the channel. These characteristics allow the use of MOTFT 12 as a sensor of the small signals generated by the change in pH as well as convenient incorporation into a matrix of structures 10, if desired. Many examples of designs and materials for MOTFT 12 can be found in, for example, U.S. Pat. No. 7,812,346, entitled “Metal Oxide TFT with Improved Carrier Mobility”, issued Oct. 12, 2010; U.S. Pat. No. 7,977,151, entitled “Double Self Aligned Metal Oxide TFT”, issued Jul. 12, 2011; and U.S. Pat. No. 8,679,905, entitled “Metal Oxide TFT with Improved Source/Drain Contacts”, issued Mar. 25, 2014, all of which are incorporated herein by reference.

Turning to FIG. 2, another example of a combined MOTFT ion sensitive and optical detection structure 60 is illustrated which includes and combines an ion sensitive MOTFT (metal oxide thin film transistor) 62 and an optical detection thin film photodiode 64. Structure 60 is fabricated on a substrate 66 which in this preferred embodiment is glass but could be other materials not necessarily transparent (unless required by the fabrication of MOTFT 62). Gate metal 68 is deposited on the surface of substrate 66 so as to extend from a central portion of structure 60, where it serves as a bottom gate for MOTFT 62, to adjacent the right-hand edge of substrate 66. A layer 69 of gate dielectric, which in this preferred embodiment is SiN but may be other insulating material, is deposited over gate metal 68. An active layer 70 of semiconductive metal oxide is deposited in overlying relationship to a bottom gate portion of gate metal 68. Source/drain contacts 72 are formed in spaced apart relationship in contact with the upper surface of active layer 70. A layer 74 of gate dielectric or insulating material is deposited on the upper surface of active layer 70 between source/drain contacts 72 and gate metal is deposited on the upper surface of gate dielectric layer 74 to define a channel in active layer 70. As will be understood, source/drain contacts 72 and top gate 76 include electrical connections (not shown) designed to electrically couple MOTFT 62 into external circuitry, such as a matrix or the like.

A thick layer 80 of insulating encapsulation material is deposited over MOTFT 62. As will be understood by artisans in the field, the insulating encapsulation material is selected to have a minimum and preferably no effect on both the electrical and chemical components of structure 60 and the subject tests. A contact layer 82 of metal is deposited on the upper surface of encapsulation layer 80 so as to extend a short distance from the right-hand edge of structure 60 to the left-hand edge where it is exposed to provide easy access as a contact terminal. A layer 84 of n+ doped a-Si is deposited over the upper surface of contact layer 82. An intrinsic or insulating layer 86 of a-Si is deposited on the upper surface of layer 84 and a layer 88 of p+ doped a-Si is deposited over a central portion of intrinsic or insulating layer 86 to form amorphous silicon photodiode 64 directly overlying MOTFT 62. An upper contact layer 90 of transparent conductive material, such as ITO or the like, is deposited over p+ a-Si layer 88 and serves as an upper contact for amorphous silicon photodiode 64. In this specific embodiment, contact layer 90 extends to the left-hand edge of structure 60 where it is exposed to provide easy access as a contact terminal.

In this specific example, a substrate for target DNA fragments is formed in overlying relationship to amorphous silicon photodiode 64 as follows. A layer 91 of transparent insulating material is deposited over contact layer 90 across the entire upper surface of structure 60. A through-hole or via 92 is formed from the upper surface of layer 91 to the upper surface of gate metal 68 and is filled with metal so that an electrical contact with the bottom gate of MOTFT 62 is formed in the upper surface of layer 91. A layer 93 of transparent conductive material (e.g. ITO or the like) is deposited over the upper surface of layer 91 so as to extend above the area encompassed by p+ a-Si layer 88. Layer 93 also extends into contact with the metal in via 91 so as to be in electrical contact with the bottom gate of MOTFT 62 and further extends to the outer edge of structure 60 where it is exposed to provide easy access as a contact terminal. A sensing layer 95 of some transparent non-conductive material, such as SiN, tantalum oxide, or the like is deposited over the upper surface of conductive layer 93 and forms a substrate for target DNA fragments 96. A wall 97 is formed on the upper surface of sensing layer 95 so as encircle the substrate and form a reservoir 94 to contain a liquid 99 containing DNA nucleotides on the upper surface of the substrate. An illumination source is provided above the substrate/sensing layer 95 and reservoir 94. All of the material between reservoir 94 and photodiode 64 is generally referred to as the ‘bottom’ of reservoir 94 for convenience.

Generally, the operation of structure 60 is the same as described above for structure 10. A labeled nucleotide carried by liquid 99 into enclosure 96 in proximity to the target DNA fragments on substrate/sensing layer 95, a fluorescence event will or will not occur when the target DNA fragments are illuminated by illumination source 98, depending on whether the labeled nucleotide is incorporated onto the target DNA strand by the polymerase enzyme. The presence or absence of fluorescence is sensed by photodiode 64 which appears as a signal between contacts 82 and 90 at the left edge of structure 60. Simultaneously, the incorporation of nucleotides onto the target fragments release hydrogen ions and produce a change in the pH of liquid 99 in enclosure 96. The change in pH is sensed by a small change in voltage on conductive layer 93 and, consequently, the bottom gate of MOTFT 62. The small change in voltage on the bottom gate acts similar to a bias so that a larger signal on the top gate is required to activate (i.e. turn ON or turn OFF) MOTFT 62. Thus, the small signal is essentially amplified which, depending upon the design and construction of MOTFT 62, can be as much as a factor of 10.

Because either structure 10 or structure 60 include an ion sensing MOTFT and a photodiode which both operate on the same nucleotide incorporation occurrence, in the incorporation of a nucleotide that is designated dark (i.e. guanine in the Illumina scheme described above) the incorporation action is verified by a pH change. Of course all other incorporation events sensed by the photodiode are also confirmed or verified by the pH sensor. It is particularly important to note that for the dual detection system to operate correctly, the photodetector and the extended gate of the ion sensing MOTFT must be contiguous (i.e. operating on the same nucleotide incorporation event).

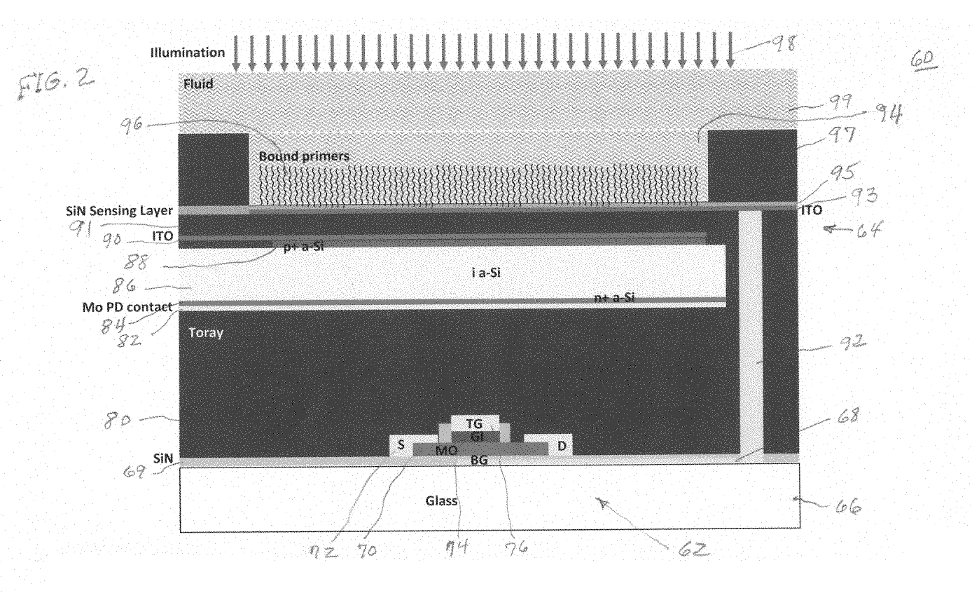

In order to further enhance or facilitate the dual detection process, two options in a biochemical bonding or linking process are illustrated in FIGS. 3 and 4. Specifically, FIG. 3 illustrates sequencing primers 100 bound to the surface of a sensing pad, such as substrate/sensing layer 95 of structure 60. Alternatively, FIG. 4 illustrates sequencing primers 100 bound to the surface of a bead, such as bead 46 in well 42 of structure 10. For simplicity of illustration, the following description illustrates sequencing primers 100 bound to a sensing pad 102 (e.g. substrate/sensing layer 95). Specifically, FIGS. 6 through 13 illustrate several steps in a chemical process for improving detection of nucleotide incorporation as a companion with the dual detection structures described above.

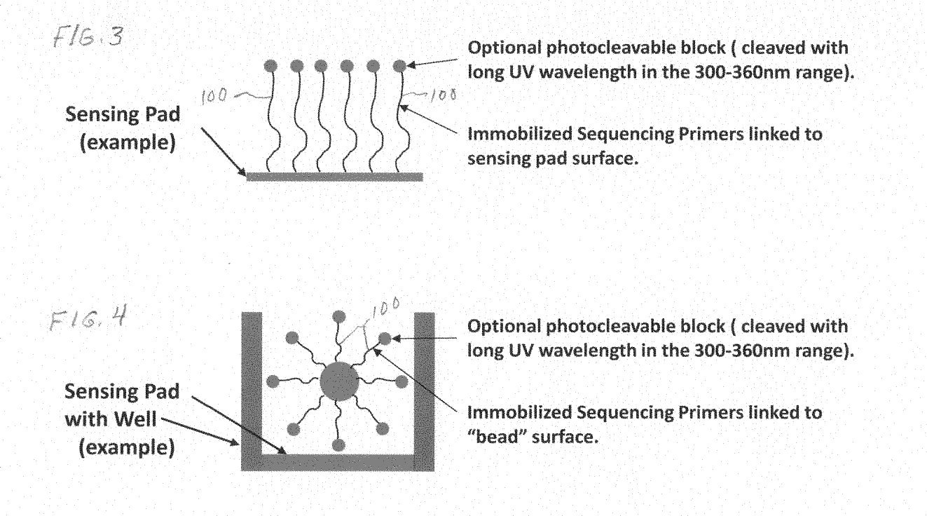

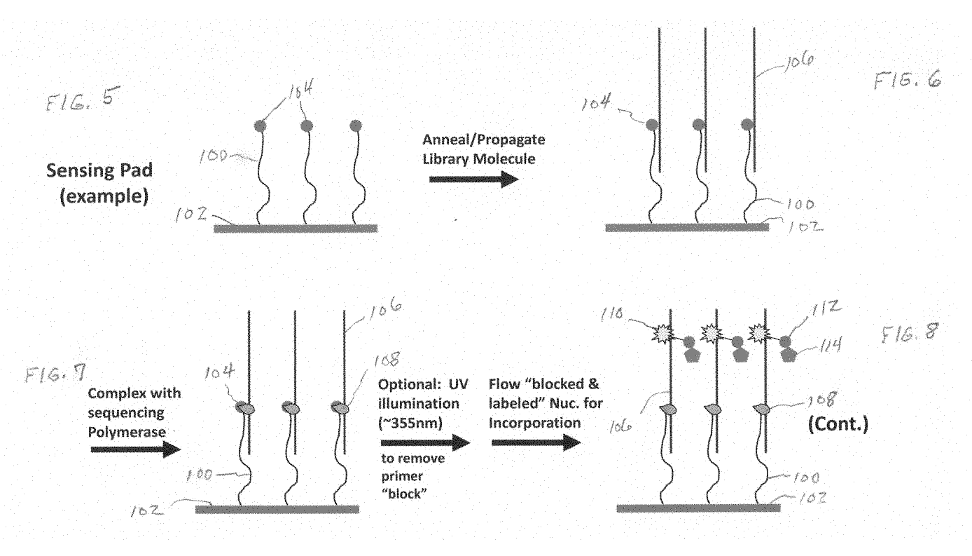

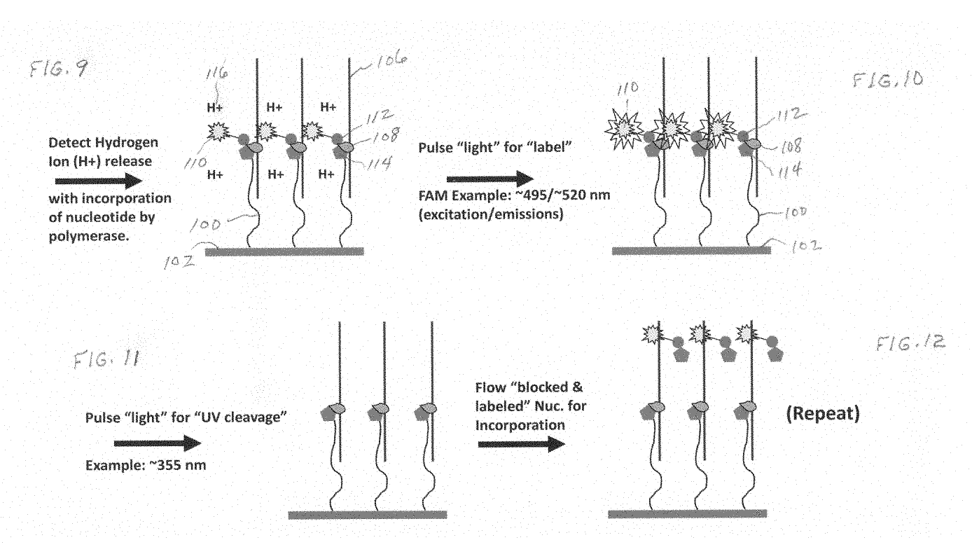

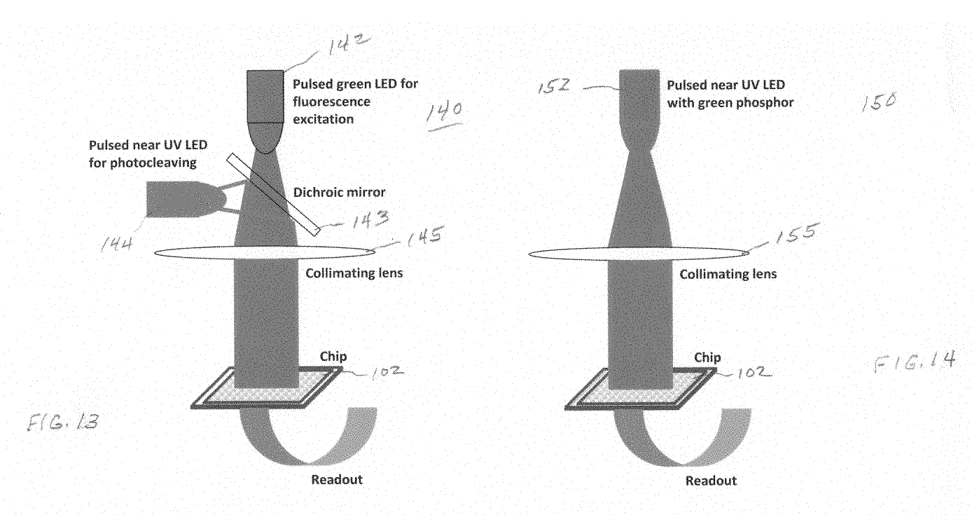

Beginning with FIG. 5, several identical sequencing primers 100 are bound to the surface of sensing pad 102. Optional photocleavable blocking molecules 104 are attached to the free end of each sequencing primer 100. Target DNA fragments 106 are then attached to sequencing primers 100, as illustrated in FIG. 6. Optional cleavable blocker 104 is complexed with a sequencing polymerase enzyme 108, as illustrated in FIG. 7. Optional blocking molecules 104 are cleaved using UV light, as illustrated in FIG. 8 which allows sequencing polymerase enzyme 108 to incorporate matching nucleotides 114 with sequencing primers 100. Matching nucleotides 114 are blocked with blocking molecules 112 and are labeled with a fluorophore 110, in the present example green dye, as illustrated in FIG. 8. Hydrogen ions 116 are released upon incorporation of nucleotides 114, as illustrated in FIG. 9. Referring to the above description of the dual detection structures, hydrogen ions 116 are detected by MOTFT 62 (in this specific example). Simultaneously, excitation light source (in this example illumination source 98) is pulsed to excite the fluorophore for the optical detection event (i.e. photodiode 64 as illustrated in FIG. 2). Excitation light is for example in a range of approximately 495 nm to approximately 520 nm (depending upon the fluorophore). The pulsing of the excitation light source is followed by pulsing of a UV light source which will cleave off the photocleavable blocking molecule 112, as illustrated in FIG. 11. UV light for the cleaving operation is for example approximately 355 nm (near UV). As illustrated by FIG. 15, the wavelengths of the excitation light and the UV cleaving light are separated sufficiently to prevent any inadvertent interaction.

With cleavable blocking molecule 112 and labeling fluorophores 110 cleaved from sequencing primers 100 and sequencing polymerase enzyme 108 still attached, as illustrated in FIG. 12, the process is ready to be repeated with the next nucleotide (starting with the step illustrated in FIG. 9). The process is repeated for each subsequent nucleotide incorporation event. Depending upon the format of the detection process, nucleotides can either be flowed sequentially or some other combination of flows may be utilized.

In an alternative embodiment to the preferred method the fluorophore is replaced by an absorbing moiety such as a gold or silver nanoparticle and the like, such that the optical absorption is increased upon the incorporation of a nucleotide. The detected intensity of the illumination by the photodiode is thus reduced by an incorporation event and increased back to the original detection level when the absorbing moiety is cleaved from the incorporated nucleotide.

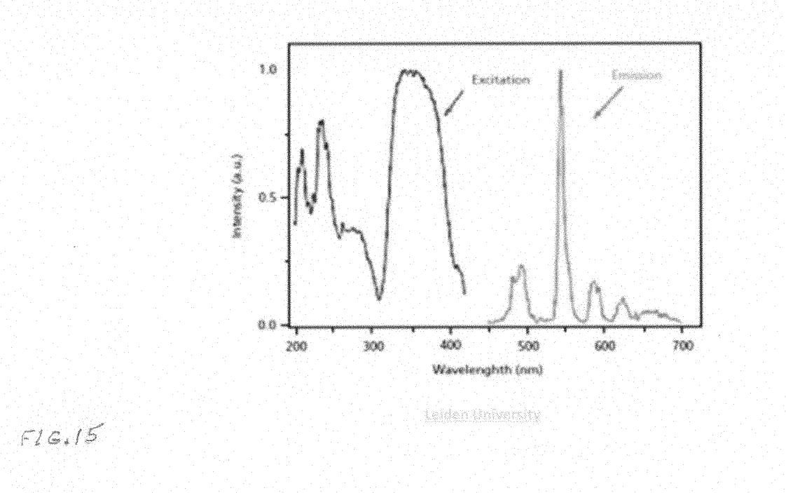

Referring to FIG. 13 an illumination device 140 is illustrated in which the fluorescent excitation and UV cleavage steps are performed in sequence or simultaneously. In device 140 a pulsed green LED 142 is positioned to direct green light through a dichroic mirror 143 onto sensing pad 102. A pulsed UV LED 144 is positioned to direct UV light onto a reflecting surface of dichroic mirror 143 which reflects the UV light onto sensing pad 102. A collimating lens 145 is positioned between dichroic mirror 143 and sensing pad 102 to collimate the green light and UV light as they are directed onto sensing pad 102.

Referring to FIG. 14, a device 150 is illustrated in which a UV LED 152 is used with a green phosphor to provide both the fluorescent excitation and UV cleavage steps simultaneously. In device 150 a narrow wavelength green phosphor is deposited on LED 152 in much the same way that white LEDs are currently made. Using a phosphor for the fluorescent excitation has the additional advantage that the phosphor has a narrow emission line (see FIG. 15) that makes it easy to filter out so as to enhance the fluorescent signal to noise ratio. Thus, device 150 simultaneously directs wavelength separated UV light and green fluorescent light through a collimating lens 155 onto sensing pad 102.

Thus, a new and improved detection process and apparatus are disclosed for DNA sequencing. The new and improved detection process and apparatus incorporates both an optical detection process and a process of detecting hydrogen ions that are released when a nucleotide is incorporated onto a target DNA fragment. The detection apparatus, or dual detector, includes a photodetector and the gate of an ion sensing MOTFT that are positioned contiguously (i.e. operating on the same nucleotide incorporation event). The dual detection can, preferably, be performed simultaneously or in the sequential steps of detecting hydrogen emission and then (once a nucleotide incorporation event is confirmed) performing the optical detection or vice versa. From the above, it will be clear that the use of combination detection of DNA incorporation events substantially improves the fidelity of the sequencing process and potentially extends the ‘read’ length of the sequencing process. Additionally, the use of blocking molecules eliminates the issues of homopolymer detection inherent in the well-known ion torrent ion selective detection process.

Various changes and modifications to the embodiment herein chosen for purposes of illustration will readily occur to those skilled in the art. To the extent that such modifications and variations do not depart from the spirit of the invention, they are intended to be included within the scope thereof which is assessed only by a fair interpretation of the following claims.

Having fully described the invention in such clear and concise terms as to enable those skilled in the art to understand and practice the same, the invention claimed is:

1. Apparatus for deoxyribonucleic acid (DNA) sequencing and more specifically for fluorescent and ion sensing of DNA nucleotide incorporation events comprising:
DNA nucleotide incorporation structure designed to have DNA target fragments or sequencing primers bonded to a surface in or on the structure for the incorporation of DNA nucleotides onto the DNA target fragments or sequencing primers, at least some of the DNA nucleotides having a fluorescent label;
a photodiode positioned adjacent the DNA nucleotide incorporation structure;
an illumination device positioned in proximity to the DNA nucleotide incorporation structure to illuminate DNA nucleotides incorporated onto the DNA target fragments or sequencing primers, the illumination device exciting the fluorescent labels when incorporation occurs and the photodiode positioned to sense the excited fluorescent labels; and
ion sensing apparatus positioned adjacent the DNA nucleotide incorporation structure including a metal oxide thin film transistor with a gate electrically coupled to receive an electrical signal indicative of ion emissions produced by the DNA nucleotide incorporated onto DNA target fragments or sequencing primers.

2. The apparatus claimed in claim 1 wherein the DNA nucleotide incorporation structure includes one of a reservoir or a well with a bottom and a sensing layer incorporated in the bottom, the sensing layer including an ion sensing element electrically coupled to the gate of the metal oxide thin film transistor.

3. The apparatus claimed in claim 2 wherein at least the bottom of the DNA nucleotide incorporation structure is transparent to light emitted by the excited fluorescent labels and the photodiode is positioned below the bottom of the DNA nucleotide incorporation structure and receives the light emitted by the excited fluorescent labels through the bottom.

4. The apparatus claimed in claim 1 wherein the metal oxide thin film transistor includes a top gate and a bottom gate with either the top gate or the bottom gate electrically coupled to receive the electrical signal indicative of ion emissions and the other of the top gate or the bottom gate connected to amplify the electrical signal.

5. The apparatus claimed in claim 1 wherein the illumination device includes a near UV LED for photocleaving and a green LED for fluorescence excitation.

6. The apparatus claimed in claim 5 wherein the near UV LED and the green LED are connected for pulsed operation.

7. The apparatus claimed in claim 6 wherein the near UV LED and the green LED are pulsed one of simultaneously or sequentially.

8. The apparatus claimed in claim 5 wherein the near UV LED and the green LED are positioned to have emissions combined into a single path directed onto the surface of the nucleotide incorporation structure.

9. The apparatus claimed in claim 1 wherein the photodiode is an amorphous silicon diode.

10. The apparatus claimed in claim 9 wherein the amorphous silicon diode includes a p+ doped amorphous silicon layer, an n+ doped amorphous silicon layer, and an undoped or intrinsic amorphous silicon layer sandwiched between the p+ and n+ doped layers.

11. A method of fabricating apparatus for deoxyribonucleic acid (DNA) sequencing and more specifically for fluorescent and ion sensing of DNA nucleotide incorporation events, the method comprising the steps of:
providing a substrate;
fabricating one of ion sensing apparatus including a metal oxide thin film transistor and an amorphous silicon photodiode on the substrate;
fabricating another of the ion sensing apparatus and the amorphous silicon photodiode adjacent the one of the ion sensing apparatus and the amorphous silicon photodiode fabricated on the substrate;
fabricating one of a reservoir and a well overlying the amorphous silicon photodiode, fabricating the one of the reservoir and the well with a transparent bottom and a sensing layer incorporated in the bottom, the sensing layer including an ion sensing element positioned to sense ion emissions in the one of the reservoir or the well, electrically coupling the sensing element to a gate of the metal oxide thin film transistor, and designing the one of the reservoir and the well to have DNA target fragments or sequencing primers bonded to a surface for the incorporation of DNA nucleotides onto the DNA target fragments or sequencing primers, at least some of the DNA nucleotides having a fluorescent label; and
providing an illumination device positioned adjacent the reservoir or the well to illuminate DNA nucleotides incorporated onto the DNA target fragments or sequencing primers, the illumination device exciting the fluorescent labels when incorporation occurs and the photodiode positioned to sense the excited fluorescent labels.

12. A method of deoxyribonucleic acid (DNA) sequencing and more specifically fluorescent and ion sensing of DNA nucleotide incorporation events, the method comprising the steps of:
providing a sensing pad and bonding sequencing primers to a surface of the sensing pad;
attaching target DNA fragments to the sequencing primers;
attaching sequencing polymerase enzymes to the target DNA fragments;
using the sequencing polymerase enzymes, incorporating complementary DNA nucleotides onto the target DNA fragments, hydrogen ions are released upon incorporation of the matching DNA nucleotides;
attaching blocking molecules to the matching nucleotides and labeling the matching nucleotides with fluorophores;
illuminating the attached and labeled target DNA fragments and sequencing primers to excite the fluorophores;
sensing the release of hydrogen ions and fluorescent emissions of the fluorophores;
cleaving the blocking molecules and the matching nucleotides from the sequencing primers; and
repeating the steps of using, attaching blocking molecules, illuminating and sensing the release of hydrogen ions and fluorescence of the fluorophores for additional sequencing events.

13. A method of deoxyribonucleic acid (DNA) sequencing as claimed in claim 12 wherein the steps of providing the sensing pad and sensing the release of hydrogen ions and the excitation of the fluorophores include providing apparatus for sensing both fluorescent and ion emissions during nucleotide incorporation events, the apparatus including an ion sensing metal oxide thin film transistor and an amorphous silicon photodiode on a common substrate, and one of a reservoir and a well overlying the amorphous silicon photodiode, both the reservoir and the well having a transparent bottom and a sensing layer incorporated in the bottom, the sensing layer including an ion sensing element positioned to sense ion emissions in the reservoir or the well, the sensing element electrically coupled to a gate of the metal oxide thin film transistor, and both the reservoir and the well having a surface that forms the sensing pad.
